(19) United States
(12) Patent Application Publication
(10) Pub. No.: US 2014/0012540 A1
(43) Pub. Date: Jan. 9, 2014

(54) APPARATUS AND METHOD FOR AUTOMATICALLY MONITORING AN APPARATUS FOR PROCESSING MEAT PRODUCTS

(71) Applicants: Michael Jurs, Neustadt (DE); Ulf Jacobsen, Bad Schwartau (DE); Henning B. Pedersen, Ikast (DK)

(72) Inventors: Michael Jurs, Neustadt (DE); Ulf Jacobsen, Bad Schwartau (DE); Henning B. Pedersen, Ikast (DK)

(73) Assignee: Nordischer Maschinenbau Rud. Baader GmbH + Co. KG, Lubeck (DE)

(21) Appl. No.: 14/006,333

(22) PCT Filed: Mar. 27, 2012
(86) PCT No.: PCT/EP12/55431

§ 371 Date: Sep. 20, 2013

(30) Foreign Application Priority Data
Mar. 28, 2011 (DE) 10 2011 015 849.9

(51) Int. Cl. G01N 33/12

(52) U.S. Cl. CPC G01N 33/12
USPC 702/173; 702/189

(57) ABSTRACT

The invention relates to an apparatus for automatically monitoring an apparatus for processing meat products, in particular fish, comprising a prediction unit for determining a yield-relevant prediction variable, the prediction unit being connected to input sensors for acquiring geometric and/or weight data of the meat product fed to the apparatus for meat processing, a yield determining unit for determining at least one yield-relevant yield variable, the yield determining unit being connected to the output sensors for acquiring geometric and/or weight data of the meat product processed by the apparatus for meat processing, and a difference unit for calculating a difference variable from the difference between one of the prediction variables and the at least one corresponding yield variable, the difference unit in each case being connected to the prediction unit and the yield determining unit. The invention further relates to a corresponding method for automatically monitoring the apparatus.

### CROSS-REFERENCE TO RELATED APPLICATIONS

This application is a National Stage Application of PCT/EP2012/055431, filed Mar. 27, 2012, which designates the United States and claims the priority of German patent application DE 10 2011 015 849.9, filed on Mar. 28, 2011, the subject matter of which is incorporated herein by reference.

### BACKGROUND

The invention relates to a method for automatically monitoring an apparatus for processing meat products, in particular fish.

The invention further relates to a monitoring apparatus for automatically monitoring an apparatus for processing meat products, in particular fish.

Such apparatuses and methods are used in various branches of the meat processing industry, in particular the fish processing or poultry processing industry, in which unprocessed or also partially pre-processed meat products are processed automatically. In principle, meat processing apparatuses can process meat products of different categories, shapes or weight ranges. The meat processing apparatus is adapted appropriately in each case for the different meat products still to be processed. For this purpose, the meat product still to be processed is measured, particularly the height, width and/or length thereof. The machine parameters of the meat processing apparatus are set according to results of these measurements. Thus, for fish processing apparatuses, the blade spacings can be set for belly blades, side blades or back blades depending on the body height of the fish still to be processed. Typically, such settings of the meat processing apparatus, such as the blade spacings for example, are called the machine parameters. If the machine parameters are set to the meat product to be processed, then the meat product is subsequently processed by the meat processing apparatus. Last but not least, the processed meat products are measured after processing for marketing. Thereby, preferably the length, width, height and/or the weight of the meat product are measured.

For improved understanding of the invention, in the following the meat product fed to a meat processing apparatus is designated as the input meat product and the meat product corresponding thereto and processed by the meat processing apparatus is denoted as the output meat product. However, due to processing, along with the meat products that are to be utilized or acquired, often further meat products also result that are not to be utilized. Subsequently, the portion of the output meat product that is intended to be utilized or acquired by means of the meat processing apparatus is designated as the meat product yield. The remaining portion of the output meat product is designated as the carrier product. The carrier product here does not necessarily serve as a carrier for the meat product yield. The carrier product can rather also comprise entrails or other parts of an animal body.

An exemplary purpose of a fish processing apparatus is to separate the filleted flesh from the fish bones of a fish body fed to a fish processing apparatus. In this case, the input meat product would be the fish body. The output meat product would be the filleted meat yielded in the process and the remaining part of the fish body. The output product is divided up into the meat product yield, namely the fish fillet, and the carrier product, namely the remaining output meat product, in particular the fish skeleton.

A method and an apparatus for determining the volume, the shape or the weight of fish or other objects are known from DE 4204843 A1. According to this, for determining the volume, shape or the weight of fish, for each fish, which is initially transported on a conveyor belt, a series of images of the contour of the fish is taken using a camera. Then, the volume of the fish, or the weight thereof, is calculated using a microprocessor on the basis of the received image data. A composite picture of the fish with many cross-sections arises from the image series from the camera, wherein the width and the maximum thickness of the respective fish are measured in each cross-section. The volume of the fish is obtained by multiplying the cross-sectional regions by the speed of the conveyor belt and the time between the individual images. The weight of the fish is obtained by multiplying the volume by the specific weight of the type of fish to be weighed.

It is thus known from the prior art to use sensors for measuring the fish products to be processed before the processing thereof, and from this sensor data to draw conclusions regarding the volume or the weight of the fish to be processed.

It is also known that the output meat product is measured in order to draw conclusions about the weight of the output meat product. However, no statement can be made as to whether the yield corresponds to that portion of the input product which could have been acquired by means of the meat processing apparatus, or whether it deviates, and to what extent. Rather, often only measured data of the input product, the output product and/or the yield are acquired.

For clarification, the problem is explained using the following example: a meat fillet (here the desired meat product yield) can be separated from the bones or fish bones of an input meat product using a meat processing apparatus. This separation occurs, however, only to a certain degree. More often than not, residual meat still remains on the bones, or fish bones, such that a complete separation occurs only in rare cases. The yield thus describes the meat product portion which is to be separated by the meat processing apparatus from the remainder of the input meat product to be processed. The absolute yield may thus be the meat product yield, in this case therefore the removed meat product. The relative yield may be the ratio of the meat product yield to the portion of input meat product which should have been utilised or obtained, in particular the maximum amount, from the input meat product.

Often, however, only relative yields of considerably below 100% are obtained. Due to the many different types of categories, shapes, lengths, sizes and/or heights of an input meat product to be processed, particularly high requirements are set for the meat processing apparatus. Not every meat processing apparatus is equally suited for processing various categories and/or shapes of meat products in the manner that results in a uniformly high, in particular relative, yield. Moreover, it is necessary to adjust the machine parameters of a meat processing apparatus to the respective meat product to be processed. In doing so, the machine parameters are subject to natural limits. Thus it is usual for fish processing apparatuses, for example, to be configured in a manner that they are only able to process fish with a body height within a predetermined body height range, with particularly good results, in the sense of yield. Now, if fish with the same body height is processed by this fish processing apparatus, it is still however possible that a different yield is attained in each case. This can be due to the fact, for example, that the fish are fish with the same body height that are narrow and long, or short and wide, or of average length and average width. A plurality of shapes are conceivable between these extremes, and these shapes exist in practice. Due to the differently formed shapes of the fish in each case, or respectively the meat product to be processed, it is often not possible for the meat processing apparatus to process the meat product to be processed in a uniformly good manner that attains the maximum yield in each case. Thus, for example, the processing blades of a fish processing apparatus cannot follow each contour of the fish skeleton of a fish to be processed. As a result, residual meat always remains attached to the fish bones. This leads to an absolute yield that is considered poor or not optimal.

Thus it is not an error of the apparatus when, for example, fish with the same body height can be processed by the same meat processing apparatus only with different relative and/or absolute yields. Therefore, it is not possible to obtain information about a fault or an incorrect setting of the meat processing apparatus solely from, in particular, the absolute or relative yield of an input meat product to be processed.

### SUMMARY

An object of the invention is to create a method and an apparatus with which the yield which can be obtained by a meat processing apparatus, in particular the relative and/or absolute yield, can be monitored.

The object is achieved by a method having the features of claim 1. In this case, initially input signals from input sensors are measured. The input sensors are sensors for acquiring geometric data and/or weight data of the input meat product fed to the apparatus for meat processing. An input signal may be understood as the signal which is emitted and/or altered by an input sensor. Thus, the input signal may be an analogue voltage characteristic. “Geometric data of the meat product” may be understood as spatially-related data of the meat product, such as the length, width, height, shape and/or external contour of the meat product. A “fed input meat product” may be understood as both a pre-processed and unprocessed meat product. Thus, a fed input meat product may be an unprocessed fish or a fish body which has already been gutted.

Moreover, the measurement of input signals from input sensors serves for determining at least one prediction variable which is relevant to yield. The determination process may also be a preferred embodiment of the invention. The prediction variable may be a variable which is relevant to yield, representing the shape, the weight and/or the volume of at least one portion of the fed input meat product. As already explained above, the yield is the meat portion which is to be separated by the meat processing apparatus from the remaining meat product. If a fed input meat product still comprises bones or a skeleton or respectively a fed fish still has fish bones or a skeleton, the meat processing apparatus or respectively the fish processing apparatus may be configured so that the actual meat is separated from the bones, the skeleton, the fish bones or the fish skeleton. In practice, a residual part of the actual meat regularly remains adhered to the bones, the skeleton, the fish bones or the fish skeleton, so that the actual meat cannot be completely separated, utilised or obtained from the remaining part of the meat. Therefore, the meat portion of the meat product which is to be separated from the remaining part of the fed input meat product by means of the meat processing apparatus may be the relevant yield. In other words, a yield-relevant prediction variable may be understood as a variable by which the yield shape, the yield weight and/or the yield volume of at least one portion of the fed input meat product may be predicted. In a particularly preferred embodiment, the yield-relevant prediction variable is the expected yield weight, at least of one portion, of the fed input meat product or fish. In a particularly preferred embodiment, the “yield-relevant prediction variable” may be understood as the expected weight, the expected geometric dimensions and/or the expected shape of the yield. It is thus preferable that the yield-relevant prediction variable does not determine the entire volume and/or the entire weight of the fed input meat product or fish, but only the part thereof which is able to be utilised.

The method according to the invention is also characterised by measuring output signals from the output sensors for acquiring geometric data and/or weight data, in particular of a corresponding meat product, preferably corresponding to the input meat product, preferably the meat product yield and for determining a yield-relevant yield variable. In this case, the determination process may also be an advantageous embodiment of the invention. Therefore, geometric data and/or weight data of the meat product yield may be acquired by means of the output sensors. An “output signal” may be understood as a signal which originates from the output sensor or has been altered thereby, such as for example an analogue voltage characteristic. So that the output sensors are able to acquire data of the meat product yield, the output sensors may be arranged in the discharge area of the meat processing apparatus. Moreover, it is possible that the output sensors are arranged downstream of the meat processing apparatus. At least one yield-relevant yield variable may be determined by means of the output signals of the output sensors. “Yield-relevant yield variable” may be understood as a variable representing the yield-relevant shape, the yield-relevant weight and/or the yield-relevant volume of the meat product yield. In a particularly preferred embodiment, the yield-relevant yield variable is the weight of the meat product yield.

Moreover, the method according to the invention is characterised by calculating a difference variable by means of a difference unit, by calculating the difference between at least one of the prediction variables and the at least one corresponding yield variable. A particularly preferred embodiment is characterised by calculating a difference between one of the prediction variables and the one corresponding yield variable. If the meat product to be processed is a fish and the body of the fish is divided up into two portions, a prediction variable may be determined for each portion of the fish body. If, moreover, by means of a fish processing apparatus, the two portions of the body of the fish are separated from the rest of the fish, the geometric data and/or the weight data of the processed and separated portions of the fish body may be acquired and two yield-relevant yield variables determined therefrom. In the cited example, in each case a prediction value and a yield value are assigned to each portion of the fish body. Thus, the calculation of the difference may take place by means of the difference unit between a prediction variable assigned to one of the portions and the yield variable assigned to the same portion, i.e. corresponding yield variables, by the difference variable being calculated between the prediction variable and the corresponding yield variable. In other words, the difference variable may be calculated so that the yield variable is subtracted from the prediction variable or the prediction variable is subtracted from the yield variable. Provided a plurality of yield-relevant prediction variables have been determined for the fed input meat product or for a portion of the fed input meat product, and/or provided a plurality of yield-relevant yield variables have been determined for the meat product yield or for a portion of the meat product yield, the difference may also be calculated between one of the prediction variables and a plurality of corresponding yield variables or between a plurality of prediction variables and one of the yield variables. Provided a plurality of prediction variables have been determined, said prediction variables may also be added to a common prediction variable. Provided a plurality of yield-relevant yield variables have been determined, said variables may also be added to a common yield-relevant yield variable.

An advantageous development of the invention provides that the prediction variable is calculated from the input signals by means of a morphology model. The morphology model may be a specific morphology model for the meat product to be processed, in particular fish. For processing fish, the corpulence factor (K factor or KF) can be considered in the morphology model. The K factor may be different for each fish and/or for each fish category. Moreover, a yield factor (AF) may be considered in the morphology model, a different yield factor being possible for each fish. A simple morphology model for a fish could be configured as follows, taking into consideration the aforementioned factors:

Yield-relevant prediction variable=(length of fish)³×KF×AF:100.

Basically, the corpulence factor and/or the yield factor can depend on the measured geometric data and/or weight data.

As the prediction variable and the yield variable in each case are yield-relevant variables, the achievable yield may be monitored by means of the resulting difference variable. Thus, the entire volume and/or the entire weight of the fed input meat product are not compared with that of the output meat product and/or meat product yield. Instead, a difference is calculated between the variables relevant thereto, namely between the yield-relevant variables. In other words, the meat processing apparatus may be decoupled or monitored independently of the parts of the fed input meat product which are not intended to be utilised by the meat processing apparatus or not intended to be separated from the remaining part of the meat product. Again, in other words, the present invention permits a monitoring of the meat processing apparatus by means of the data of that part or portion of the fed input meat product which is intended to be separated by the meat processing apparatus from the remaining part and/or portion of the fed input meat product. Thus, an objective monitoring of the meat processing apparatus may take place using the yield-relevant parts and/or portions of the fed input meat product and the meat product yield.

In addition to an achievable yield, information about the efficiency of the meat processing apparatus may also be obtained from the difference variable. Thus, the efficiency of the meat processing apparatus may be considered as a variable which is dependent on the difference variable in a linear manner. In an advantageous embodiment of the invention, the level of efficiency may also be emitted optically and/or acoustically, in particular by means of an output apparatus.

Moreover, it may be advantageous if information is not only obtained about the efficiency of the apparatus for meat processing, but also whether the meat processing apparatus might have processed the meat product more efficiently or whether the meat processing apparatus is set up incorrectly and/or whether the meat processing apparatus in principle operates incorrectly.

A preferred embodiment of the invention is characterised in that the prediction variable is calculated from the input signals and machine parameters by means of a morphology model and a machine model. In this case, the morphology model may be configured as explained above. In principle, machine parameters may be understood as all parameters for adjusting the meat processing apparatus. With a fish processing apparatus the parameters could be, for example, the parameters which determine the blade-distance of belly blades, side blades and/or back blades. If, for example, the external diameter (DA) of the fish is determined by the input sensors of a fish to be processed, and the external diameter of the fish skeleton (DI) is determined by a morphology model and if the belly blades of the fish processing apparatus have a specific blade-distance (MA), then a simple machine model can be configured as follows:

$$MF = \{ \begin{matrix}( \frac{DA - MA}{DA - DI} ) & for DA > MA > DI \\ 0 & otherwise\end{matrix} \}$$

where MF represents the machine factor. The machine factor can also have influence when taking into consideration the prediction variable. The prediction variable can therefore be determined from the input signals by means of the morphology model and the machine model, as follows:

Prediction variable=(length offish)³×KF×AF×MF:100.

Due to the input signals and/or geometric data and/or weight data of the fed input meat product and by means of a morphology model and machine model, the information that describes the adaptation of the meat processing apparatus to the meat product to be processed can also influence the determination of the prediction variable.

A further advantageous embodiment of the invention is characterised in that a corresponding value from a database is assigned to the prediction variable depending on the input signals or the input signals and machine parameters, prediction variables being stored in the database depending on input signals or input signals and machine parameters. In a particularly simple embodiment, the database has a plurality of different combinations of input signals or input signals and machine parameters, a corresponding prediction variable being stored in the database for each combination of input signals or for each combination of input signals and machine parameters. If input signals of the input sensors are measured for acquiring geometric data and/or weight data of the input meat product fed to the apparatus and if preferably the machine parameters which are provided for the meat product are taken into consideration, in order to process this, a comparison may be made with the values of the input signals or input signals and machine parameters from the database so that exactly the same parameters are found in the database or that the data set of the input signals or input signals and machine parameters is found in the database which, in particular in the mean value, has the least deviation. The variable which is stored in the database corresponding to the determined data set for the prediction variable may then be assigned to the prediction variable. In other words, the input signals or the input signals and the machine parameters may be used in order to read from a database a prediction variable corresponding to the input signals or input signals and machine parameters.

A further advantageous embodiment of the invention is characterised by determining a comparison variable by comparing the difference variable with a reference variable. The reference variable may optionally be predetermined, calculated or assigned. The “comparison” may be understood as calculating a difference or ratio. If the reference variable is predetermined, for example, by a value of 10 and a difference variable of, for example, 5 is determined from the yield-relevant prediction variable and the yield-relevant yield variable, by calculating the difference, the difference variable may be compared with the predetermined reference variable. When calculating the difference, the comparison variable would be 5. When calculating the ratio of the difference variable to the reference variable, the comparison variable would be 0.5. A statement about the difference variable may be obtained by comparing the difference variable with the reference variable. The reference variable may, therefore, be taken into consideration as a measurement or a benchmark for the difference variable. If, therefore, the comparison variable is determined by calculating the difference between the difference variable and the reference variable, the statement can be made that the apparatus for fish processing has a high yield when the comparison variable is low, an average yield when the comparison variable is average and a low yield when the comparison variable is high. In this case, a comparison variable may be regarded as low when it is between 0 and 5, as average when between 5 and 10 and high when greater than 10. These values are only to be understood by way of example as the difference variable may vary widely according to the weight, volume, length, height and/or width of the meat product yield. If the difference variable of, for example, 5 (grams) is regarded as particularly low for a meat product to be processed with a total weight of 2 (kilograms), the same difference variable may be regarded as high when the meat product to be processed has, for example, a total weight of 70 (grams). Moreover, the difference variable may also depend on how far the processing apparatus may be adjusted to the meat product to be processed. If, for example, the minimum blade spacing for the belly blades in a fish to be processed is greater than or considerably greater than the external diameter of the fish skeleton in the belly region, the fish processing apparatus is not able to separate the entire belly flesh from the fish skeleton, even when it is correctly set up and/or operates optimally.

The question is thus also raised here of how a monitoring device may be configured in order to be able to provide information about whether the meat processing apparatus could process the meat product more efficiently, whether the meat processing apparatus is set up incorrectly or not optimally or whether the meat processing apparatus in principle operates incorrectly.

An advantageous embodiment of the invention is characterised by calculating the reference variable from the input signals and the machine parameters by means of the machine model and morphology model. By considering the morphology model and the machine model, information may be provided as to how well the apparatus or the machine settings, in particular determined by the machine parameters, are suited to the fish to be processed. By means of the input signals and the morphology model, information may be obtained about the structure, the design, the yield-relevant prediction variable and further geometric data and/or weight data of the fish to be processed. If, therefore, the input signals and the machine parameters as well as a morphology model and a machine model are known, it is possible to calculate therefrom how great the difference is between the yield-relevant prediction variable and the yield-relevant yield variable, the calculated difference being understood as the reference variable.

A further advantageous embodiment of the invention is characterised in that a corresponding value from, in particular, a further database is assigned to the reference variable depending on the input signals and machine parameters or depending on the prediction variable and machine parameters, reference variables depending on input signals and machine parameters or depending on prediction variables and machine parameters being stored in the database. The method of assigning a value from a database has already been described above. This also applies to the last-mentioned database by considering the variables and parameters relevant to said database.

In other words, an anticipated difference variable may be understood by “reference variable”. This is because into the calculated or assigned reference variable the information might be incorporated which result in a specific difference variable. If, for example, the belly blades are not able to separate the entire belly flesh of a fish, as the fish processing apparatus is entirely unsuitable therefor, the fish processing apparatus is not at fault and it is not a faulty setting of the fish processing apparatus. Said analogue and/or further data may be considered for calculating or assigning the reference variable. By comparing the reference variable with the difference variable, it is possible to obtain objective information about the status of the apparatus.

A further advantageous embodiment of the invention is characterised by generating a control signal when the difference variable reaches, exceeds or falls below a tolerance threshold of the reference variable. In this case, the reference variable may have a tolerance range. The tolerance range may be limited by an upper tolerance threshold and/or a lower tolerance threshold. The lower tolerance threshold may have a value which is lower than the reference variable. The upper tolerance threshold may have a value which is higher than the reference variable. In other words, the reference variable may be assigned tolerance variables which are lower or higher than the reference variable. By the tolerance range or the tolerance thresholds, the range around a reference variable may be determined which is tolerated for the difference variable. The control signal may be an analogue or digital signal. In particular, it may be a voltage jump in an analogue signal.

The meat product processed by the apparatus for meat processing is often divided into different portions or parts. Thus, for example, the meat of a fish body may be divided into three portions or parts. These parts of the processed fish are then preferably separately and/or successively transported away from the apparatus for fish processing. In particular, in order to determine a difference variable between corresponding prediction variables and yield variables, it may be expedient to identify corresponding portions of the fed input meat product. An advantageous embodiment of the invention is characterised by identifying a portion of the fed input meat product from the input signals by means of a morphology model. The identification may also or alternatively take place by means of a machine model. In this case, it is advantageous if the geometric boundaries of the respective portion correspond to the geometric outer edges of the meat product yield. If, for example, a meat product is cut into three strips of uniform width, the portions of the fed meat product may be identified so that the fed input meat product is also divided into three portions of uniform width, not physically but in particular virtually, apparently and/or functionally. The portions identified in this manner of the fed input meat product correspond in their geometric shape at least approximately to the geometric shape of the meat product yield. Up to 1, 2, 3, 5, 7, 10, 12 or 15% of the difference in length between the identified portions and the processed portions may be acceptable. If the individual portions of the fed input meat product are not identified, it may be expedient for a difference to be calculated between the prediction variable of the fed input meat product and the sum of the corresponding yield variables of the meat product yield.

A further advantageous embodiment of the invention is characterised in that a portion of the fed input meat product is identified by means of, in particular, a further database depending on the input signals. Portion data of meat products depending on input signals may be stored in said database. The portion data may be data or values which represent, determine or make determinable the corresponding geometry and/or external contour. In other words, the portion data may be values and/or data representing a portion.

According to an embodiment of the invention the value may be assigned to a portion of the fed input meat product, said value being stored in the database for the corresponding measured input signal. Moreover, the assignment of a value from a database may take place in a manner similar to the already explained methods.

The object mentioned hereinbefore is also achieved by a monitoring apparatus for automatically monitoring an apparatus for processing meat products, in particular fish, by a prediction unit for determining a yield-relevant prediction variable, the prediction unit being connected by means of a data connection to input sensors for acquiring geometric data and/or weight data of the input meat product fed to the apparatus for meat processing, a yield determining unit for determining at least one yield-relevant yield variable, the yield determining unit being connected to the output sensors for acquiring geometric data and/or weight data of the meat product yield by means of a further data connection, and a difference unit for calculating a difference variable from the difference between, in particular, at least one of the prediction variables and the at least one corresponding yield variable, the difference unit being connected to the prediction unit by a further data connection and the difference unit being connected to the yield determining unit by a further data connection.

### BRIEF DESCRIPTION

Further expedient and/or advantageous features and/or developments of the invention are revealed from the sub-claims and the description. A particularly preferred embodiment is described in more detail with reference to the accompanying drawings. The drawings show:

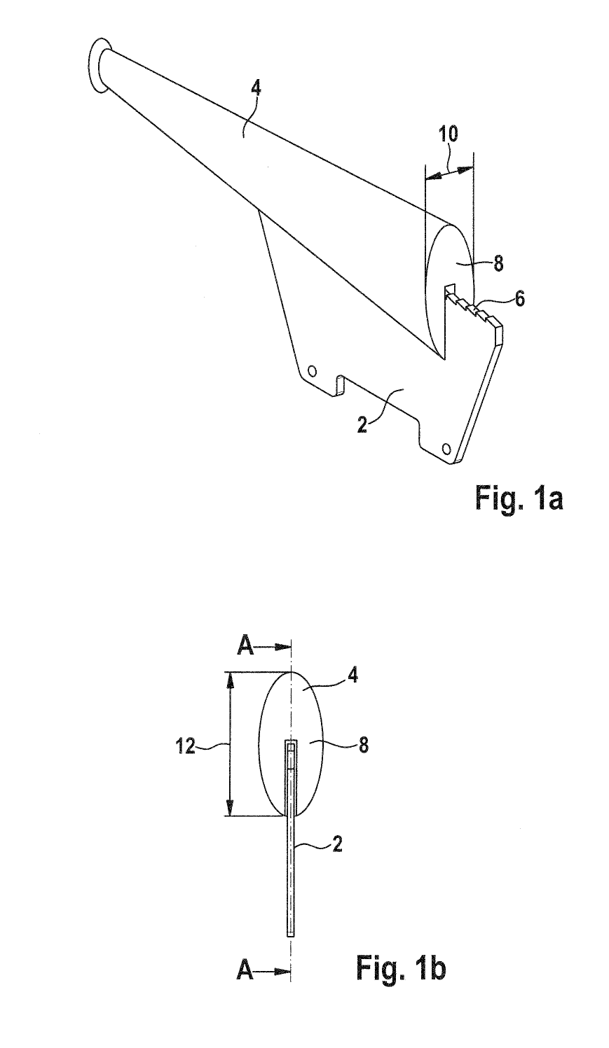

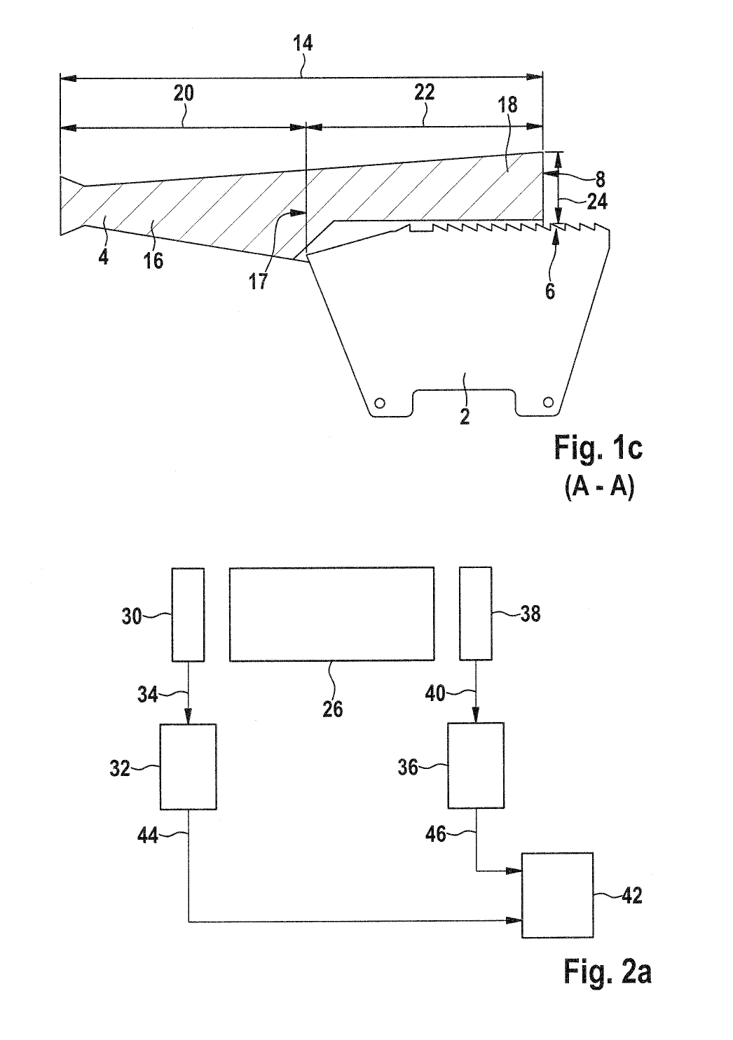

FIG. 1A is a view of a transport saddle with a fish body in a perspective view,

FIG. 1B is a view of a transport saddle with a fish body in a front view,

FIG. 1C is a view of a transport saddle with a fish body in a sectional side view,

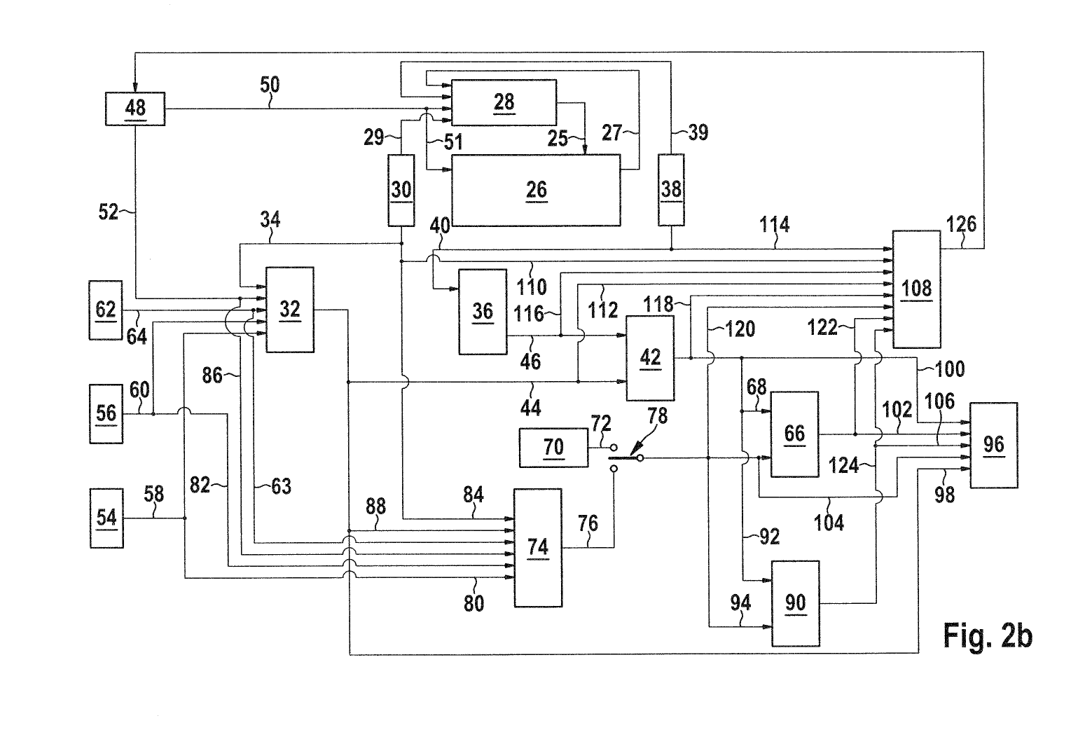

FIG. 2A is a schematic view of a block diagram of a monitoring apparatus for automatically monitoring a meat processing apparatus, and

FIG. 2B is a schematic view of a block diagram of a monitoring apparatus for automatically monitoring a meat processing apparatus with further advantageous embodiments.

### DETAILED DESCRIPTION

For improved understanding of the invention initially a transport saddle 2 and a model of a fish body 4 are shown in FIG. 1. The fish body 4 in this case is fastened to the transport saddle 2. To this end, the transport saddle 2 has transport teeth 6 on the upper edge thereof. By means of the transport saddle 2, the fish body 4 is fed to a meat processing apparatus, conveyed therein and transported away from the fish processing apparatus. The fish body 4 at the cutting edge 8 thereof has a product width 10. The product width 10 of the fish body 4 may be detected by the input sensors of a monitoring apparatus.

In FIG. 1B, the front view of the cutting edge 8 of the fish body 4 is shown, the fish body 4 being fastened to the transport saddle 2. The fish body 4 has a specific height 12 on the cutting edge 8. Moreover, in FIG. 1B the section A-A is shown.

According to the section A-A in FIG. 1C the view of the cutting plane A-A is shown. The transport saddle 2 as well as the lateral sectional view of the fish body 4 are shown. The fish body has in this case an overall length 14. Moreover, shown in FIG. 1C are the portions 16 and 18 of the fish body 4 into which the fish body 4 is intended to be divided up by the fish processing apparatus. The portion 16 of the fish body 4 has in this case a length 20 which is shorter than the overall length 14 of the fish body 4. Accordingly, the other portion 18 of the fish body 4 has a length 22 which is also shorter than the overall length 14 of the fish body 4. The fish body 4 is fastened to the transport saddle 2 such that the cutting edge 8 protrudes by a distance 24 over the transport teeth 6 of the transport saddle 2.

The fish body 4 may form the basis of a morphology model. With an external diameter (D₁) of the front cutting edge 8, an external diameter (D₂) on the other portion boundary 17 and a portion length (L) 22, the portion has a volume of

$$V = \frac{L * Π}{3} * ( {( \frac{{D}_{1}}{2} )}^{2} + ( \frac{{D}_{1} * {D}_{2}}{4} ) + {( \frac{{D}_{2}}{2} )}^{2} )$$

The weight is determined from the product of the specific thickness of the fish and the volume.

In FIG. 2A, a block diagram of a monitoring apparatus for automatically monitoring an apparatus for processing meat products, in particular fish, is shown. The apparatus for processing meat products, in particular fish, may also be denoted as the meat processing apparatus 26. The at least one input sensor 30 serves for acquiring geometric data and/or weight data of the input meat product fed to the meat processing apparatus 26. In this case the meat product is detected by the input sensors 30, preferably on a transport saddle. For evaluating the geometric data and/or weight data, a prediction unit 32 is connected by means of a data connection 34 to the at least one input sensor 30. A prediction unit 32 in principle may, in particular, be exclusively adapted and/or configured to determine a yield-relevant prediction variable.

In principle, a data connection 34 between the prediction unit 32 and at least one input sensor 30 may be any type of data connection. This also applies to the data connections cited below. A data connection may, in particular, be a wired, a radio and/or a network connection.

Moreover, in FIG. 2A, a yield determining unit 36 is shown for determining at least one yield-relevant yield variable. The yield determining unit 36 is connected by means of a further data connection 40 to the at least one output sensor 38 for acquiring geometric data and/or weight data of the meat product processed by the meat processing apparatus 26. The yield determining unit 36 may, in particular, be exclusively configured and/or adapted in order to determine yield-relevant yield variables.

Moreover, in FIG. 2A a difference unit 42 for calculating a difference variable from the difference of one of the prediction variables and the at least one corresponding yield variable is shown. For transmitting the yield variable determined by the yield determining unit 36 to the difference unit 42, the difference unit 42 is connected by a further data connection 46 to the yield determining unit 36. By means of the data connection 46 between the difference unit 42 and the yield determining unit 36, the difference unit 42 is able to refer to the respective yield variable for the calculation. Moreover, the difference unit 42 is connected to the prediction unit 32 by a further data connection 44. By means of this connection, the prediction variable determined by the prediction unit 32 may be transmitted to the difference unit 42. The difference unit 42 may thus refer to the prediction variable for calculating the difference variable by means of the data connection 44.

Further advantageous embodiments and details of the invention are shown in FIG. 2B. In this case, reference is made to the embodiments of FIG. 2A, as FIG. 2A forms the basis of FIG. 2B. Thus, also in FIG. 2B, the meat processing apparatus 26, the input sensor 30, the output sensor 38, the prediction unit 32, the yield determining unit 36, the difference unit 42 as well as the data connections 34, 40, 44 and 46 are shown. The structural and/or functional connections between the apparatus, the sensors, the units and the data connection in this case correspond to the connections described in FIG. 2A.

For improved understanding of the invention, in addition to the meat processing apparatus 26 a control and regulating unit 28 is also shown. The meat processing apparatus 26 may be connected to the control and regulating unit 28, in order to control or to regulate the meat processing apparatus 26. To this end, in each case the input sensor 30 may be connected by means of a data connection 29, the output sensor 38 may be connected by means of a data connection 39 and further sensors (not shown) of the meat processing apparatus 26 may be connected by means of a data connection 27 to the control and regulating unit 28. Moreover, the control and regulating unit 28 may be connected to the meat processing apparatus 26 by means of a further data connection 25 for transmitting control and/or regulating signals.

An advantageous embodiment of the invention is characterised in that the input sensors 30 measure the length, the height, the width, the diameter, the volume and/or the weight of the fed input meat product, in particular in a contactless manner. To this end, the input sensors 30 may be measured mechanically, inductively, capacitively, optically, by means of ultrasound, by means of radar and/or by angular determination. The measurement may also take place on the moving meat product. A particularly simple embodiment of the input sensors 30 is characterised in that a light barrier arrangement comprising a plurality of light barriers is arranged transversely to the feed direction of the meat product to be fed. The length, the height and/or the width of the fed input meat product may be determined thereby, in each case the time of the light beam passed through by the fed input meat product being able to be evaluated.

A further advantageous embodiment of the invention is characterised in that the output sensors 38 measure the length, the height, the width, the diameter, the volume and/or the weight of the meat product yield, in particular using contacts. The measurement of the output sensors 38 may take place mechanically, inductively, capacitively, piezo-electrically, optically, by means of ultrasound, by means of radar, by means of strain gauge and/or by angular determination. A particularly simple embodiment is characterised in that the weight of the meat product yield is measured by a discharge apparatus. The discharge apparatus may have a weight measuring unit which, for example, measures the weight of the processed meat product transported by the discharge apparatus by means of strain gauge, inductively and/or capacitively.

The settings of the meat processing apparatus 26 may be determined by machine parameters. Said machine parameters may be stored in a machine parameter memory 48. In order to control and/or regulate the meat processing apparatus 26, the control and/or regulating unit 28 may be connected by means of a further data connection 50 to the machine parameter memory 48. Moreover, the meat processing apparatus 26 may be connected by means of a data connection 51 to the machine parameter memory 48.

Moreover, the monitoring apparatus may be configured so that the machine parameter memory 48 is connected by means of a further data connection 52 to the prediction unit 32. By means of said data connection, machine parameters may be transmitted from the machine parameter memory 48 to the prediction unit 32. In other words, the prediction unit 32 may refer to the machine parameters of the machine parameter memory 48 by means of the data connection 52. The prediction unit 32 may also be configured so that it has a morphology model and/or a machine model. It is, however, also possible that a morphology model memory 54 is connected by means of a data connection 58 to the prediction unit 32. Moreover, it is possible that a machine model memory 56 is connected to the prediction unit 32 by means of a further data connection 60. By the respective data connection, the prediction unit 32 may refer to the morphology model and/or to the machine model, in order to calculate the prediction variable from the input signals or from the input signals and the machine parameters.

Moreover, the monitoring apparatus may have a database 62. Prediction variables may be stored in the database 62 depending on input signals or input signals and machine parameters. By means of a further data connection 64, the database 62 may be connected to the prediction unit 32. The prediction unit 32 thus has access to the data of the database 62 by means of the data connection 64. The prediction unit 32 may be adapted to assign a corresponding value from the database 62 to the prediction variable depending on the input signals or the input signals and the machine parameters. The prediction unit 32 has access to the input signals via the data connection 34 between the prediction unit 32 and the at least one input sensor 30. The prediction unit has access to the machine parameters by the data connection 52 between the prediction unit 32 and the machine parameter memory 48.

Moreover, the monitoring apparatus may have a comparison unit 66. By means of the comparison unit 66, a comparison variable may be determined by comparing the difference variable with an optionally predetermined, calculated or assigned reference variable. To this end, the comparison unit 66 is connected by a further data connection 68 to the difference unit 42. By means of said data connection the comparison unit 66 has access to the difference variable of the difference unit 42. A predetermined reference variable may be stored in a reference variable memory 70. Between the reference variable memory 70 and the comparison unit 66, a further data connection 72 may be formed.

The monitoring apparatus may also have a reference variable determining unit 74. The reference variable determining unit 74 may also be connected by means of a further data connection 76 to the comparison unit 66. Moreover, a switch 78 may be provided which optionally connects the comparison unit 66 with the reference variable memory 70 or the reference variable determining unit 74. The comparison unit may preferably be configured and/or adapted exclusively for this, in order to determine a comparison variable by comparing the difference variable with the reference variable.

The reference variable determining unit 74 may have a further machine model and/or a further morphology model. The machine model and/or the morphology model in this case is preferably the same machine model and/or morphology model as preferably comprised by the prediction unit 32. It is also possible that the reference variable determining unit 74 is connected by means of a further data connection 80 to the morphology model memory 54. By means of the data connection 80, the reference variable determining unit 74 has access to the morphology model. Moreover, the reference variable determining unit 74 may be connected by means of a further data connection 82 to the machine model memory 56. By means of said data connection 82, the reference variable determining unit 74 has access to the machine model.

The reference variable determining unit 74 is, in particular exclusively, adapted and/or configured for calculating the reference variable from the input signals of the at least one input sensor 30 and the machine parameter by means of the machine model and/or the morphology model. The at least one input sensor 30 may be connected by a further data connection 84 to the reference variable determining unit 74. By means of this data connection, the reference variable determining unit 74 may refer to the input signals of the at least one input sensor 30. Also, the machine parameter memory 48 may be connected by a further data connection 86 to the reference variable determining unit 74. By means of said data connection, the reference variable determining unit 74 may refer to the machine parameters. In a further embodiment, the reference variable determining unit 74 may be adapted and/or configured to calculate the reference variable from the prediction variable and the machine parameters by means of the machine model. The reference variable determining unit 74 may be connected by a further data connection 88 to the prediction unit 32. By means of this data connection, the reference variable determining unit 74 may refer to the prediction variable of the prediction unit 32. The reference variable determining unit 74 may, in particular exclusively, be adapted and/or configured to calculate the reference variable from the input signals and/or from the prediction variable and the machine parameters.

The reference variable determining unit 74 may be adapted and/or configured to assign a corresponding value from, in particular, a further database to the reference variable depending on the input signals, the prediction variable and/or the machine parameters. The database may be the aforementioned database 62. To this end, the reference variable determining unit 74 may be connected by means of a further data connection 63 to the database 62. Reference variables depending on input signals, prediction variables and/or machine parameters may be stored in the database, in particular in the database 62. In each case the at least one input sensor 30 may be connected by the data connection 84, the prediction unit 32 may be connected by the data connection 88 and/or the machine parameter memory may be connected by the data connection 86 to the reference variable determining unit 74. The reference variable determining unit 74 thus has access to the corresponding signals or variables of the prediction unit 32 of the at least one input sensor 30, the database 62 and/or the machine parameter memory 48.

The monitoring apparatus may comprise a control signal determining unit 90 for generating a control signal. The control signal determining unit 90 may preferably exclusively be adapted and/or configured to generate a control signal. Moreover, the control signal determining unit 90 may be adapted and/or configured such that a control signal is generated when the difference variable reaches, exceeds or falls below a tolerance threshold of the reference variable. The tolerance variable in this case may be a predetermined tolerance variable. The tolerance variable may also be a variable dependent on the reference variable. Thus, the upper tolerance threshold, for example, may be 5% greater than the reference variable. The lower tolerance threshold may, for example, be 5% lower than the reference variable. Thus this would produce a tolerance range of 10% around the reference variable. Alternative thresholds and/or ranges for the tolerance are also possible. The tolerance, in particular the thresholds and/or ranges thereof, may be stored in a tolerance memory. Moreover, the tolerance may also be predetermined and/or determined externally. The signal determining unit 90 may be adapted and/or configured for calculating the difference. Moreover, the control signal determining unit 90 may be configured and/or adapted for comparing the difference variable with at least one of the tolerance thresholds. The control signal determining unit 90 is connected by a further data connection 92 to the difference unit 42. By means of this data connection, the difference variable may be transmitted to the control signal determining unit 90. In other words, the control signal determining unit 90 has access to the difference variables of the difference unit 42. The control signal determining unit 90 may be connected by a further data connection 94 to the reference variable memory 70 or the reference variable determining unit 74. The control signal determining unit thus has access to the reference variable.

The monitoring apparatus may also have an output unit 96 for the acoustic and/or optical output of the prediction variable, the difference variable, the reference variable, the comparison variable and/or the control signal. In other words, an advantageous embodiment of the invention may be characterised in that the prediction variable, the difference variable, the comparison variable and/or the control signal may be output in an optical and/or acoustic manner. The output unit 96 may be provided to this end. The output unit 96 may have a loudspeaker. The output unit 96 may have a display screen and/or lighting means. The prediction unit 32 may be connected by a further data connection 98 to the output unit 96. The difference unit 42 may be connected by a data connection 100 to the output apparatus 96. The comparison unit 66 may be connected by a further data connection 102 to the output device 96. The reference variable determining unit 74 may be connected by a further data connection 104 to the output unit 96. The control signal determining unit 90 may be connected by a further data connection 106 to the output unit 96. By these data connections, the output unit 96 has access to the corresponding variables or signals of the respective unit.

A further advantageous embodiment of the invention is characterised by determining at least one machine reference parameter by means of the input signals, the prediction variable, the output signals, the yield variable, the difference variable, the comparison variable and/or the control signal. In principle, an apparatus for meat processing 26 may have a plurality of machine parameters. By means of the machine parameters, for example, the blade spacings of a fish processing apparatus may be determined. Moreover, by the input signals and/or by the prediction variable information may be obtained about the geometric data and/or weight data of the fish and/or meat product to be processed. If, for example, a short and wide fish is to be processed by a fish processing apparatus 26, it may be necessary to increase the blade spacings of a belly blade. To this end, it is necessary that the corresponding parameter, associated with the spacing of the belly blades, is accordingly altered, in particular increased. Thus, for example the reference parameter for the blade spacing of the belly blade may be determined from the input signals and/or prediction signals.

In particular, when meat products or fish with the same and/or similar external contour are to be processed, it may be expedient to use information measured and/or determined by the already processed meat products and/or fish so that the processing of the meat products and/or fish still to be processed is improved. It may thus be expedient to determine a machine reference parameter not only by means of the input signals or the prediction variable. It may also be expedient to determine a machine reference parameter by means of the output signals, the yield variable, the difference variable, the comparison variable and/or the control signal. This may be advantageous, in particular, for the difference variable. Thus a machine reference parameter could be determined and/or calculated, by a variable dependent in a linear manner on the difference variable being added to the machine parameter. In a quite particularly simple case, the machine reference parameter is calculated by the difference variable being added to the corresponding machine parameter. Corresponding determination methods and/or apparatuses may also apply or be provided for the output signals, the yield variable, the comparison variable and/or the control signal.

The monitoring apparatus may have a parameter determining unit 108 for determining at least one machine reference parameter by means of the input signals, the prediction variable, the output signals, the yield variable, the difference variable, the reference variable, the comparison variable and/or the control signal. The parameter determining unit 108 may preferably be configured and/or adapted exclusively for determining at least one machine reference parameter. The at least one input sensor 30 may be connected by a further data connection 110 to the parameter determining unit 108. The prediction unit 32 may be connected by a further data connection 112 to the parameter determining unit 108. The at least one output sensor 38 may be connected by a further data connection 114 to the parameter connecting unit 108. The yield variable determining unit 36 may be connected by a further data connection 116 to the parameter determining unit 108. The difference unit 42 may be connected by a further data connection 118 to the parameter determining unit 108. The reference variable determining unit 74 may be connected by a further data connection 120 to the parameter determining unit 108. The comparison unit 66 may be connected by a further data connection 122 to the parameter determining unit 108. The control signal determining unit 90 may be connected by a further data connection 124 to the parameter determining unit 108. By means of the data connections with the parameter determining unit 108 said parameter determining unit has access to the corresponding variables and/or signals of the units and/or sensors connected thereto.

A further advantageous embodiment of the invention is characterised by the replacement of at least one machine parameter by the at least one corresponding machine reference parameter. If, for example, a machine reference parameter is determined for the blade spacing of the belly blade, before processing the fish to be correspondingly processed, this may be stored instead of the corresponding machine parameter in a machine parameter memory 48. In other words, the machine reference parameter may replace the corresponding machine parameter. Also a plurality of machine reference parameters may be determined by means of the above-mentioned signals or variables, which in each case replace the corresponding machine parameters and/or are stored in a machine parameter memory 48 instead of the corresponding parameters.

The parameter determining unit 108 may be adapted and/or configured for replacing at least one machine parameter from the machine parameter memory 48 by the corresponding at least one machine reference parameter. The machine parameter memory 48 may be connected to the parameter determining unit 108 by a further data connection 126. By means of this data connection, the parameter determining unit has access to the machine parameter memory 48.

A further advantageous embodiment of the invention is characterised by updating at least one of the databases 62 by the storage of variables belonging to the database 62, in particular input signals, prediction variables, output signals, yield variables, difference variables, reference variables, comparison variables, control signals and/or machine parameters. If, for example, during the processing of fish the input signals are measured from a fed fish, prediction variables are determined therefrom and after the processing thereof, output signals measured and in turn yield variables determined therefrom, difference variables determined from the corresponding prediction variables and/or yield variables, which in each case are added to the reference variables determined from the prediction variables and/or yield variables, optionally to determine therefrom comparison variables and/or control signals, the apparatus having processed the fish according to the settings according to the machine parameters, this forms for example a data set of associated variables. For each fish to be processed, associated variables are determined. Thus a plurality of data sets may exist. These may be gradually stored in a database 62. If data sets are determined which correspond to one or more variables of the data set of a data set stored in the database 62, for example depending on the difference variable and/or comparison variable and/or control signal, the determined, in particular new, data set may replace the data set already stored in the database 62. Otherwise, the already stored data set may remain in the database 62. It is also possible that both the already stored data set and the new data set is stored in the database 62. Moreover, it is possible that the aforementioned databases are integrated in a common database 62. Thus the databases may be a single database.

The parameter determining unit 108 may update the machine parameters with at least one machine reference parameter. In particular, the parameter determining unit is adapted and/or configured to replace the machine parameter from the machine parameter memory 48 by the machine reference parameter, which has the greatest similarity to and/or the smallest difference from the machine reference parameter. Alternatively, the machine reference parameter may be additionally stored in the machine parameter memory 48. In particular, a machine parameter set with a plurality of individual parameters may be understood by “machine parameter”.

1. Method for automatically monitoring an apparatus for processing meat products, in particular fish, characterized by the following steps:
a. measuring input signals from input sensors for acquiring geometric and/or weight data of an input meat product fed to the apparatus and for determining at least one yield-relevant prediction variable,
b. measuring output signals from output sensors for acquiring geometric and/or weight data of a meat product yield and for determining at least one yield-relevant yield variable, and
c. calculating a difference variable by means of a difference unit by calculating a difference between one of the prediction variables and the at least one corresponding yield variable.

2. Method according to claim 1, characterized in that the prediction variable is calculated from the input signals by means of a morphology model or from the input signals and machine parameters by means of a morphology model and a machine model.

3. Method according to claim 1, characterized in that a corresponding value from a database is assigned to the prediction variable depending on the input signals or the input signals and machine parameters, prediction variables being stored in the database depending on input signals or input signals and machine parameters.

4. Method according to claim 1, characterized by determining a comparison variable by comparing the difference variable with a reference variable which is optionally predetermined, calculated or assigned.

5. Method according to claim 1, characterized by calculating the reference variable from the input signals and the machine parameters by means of the machine model and/or the morphology model.

6. Method according to claim 1, characterized in that a corresponding value from a database is assigned to the reference variable depending on the input signals and machine parameters or the prediction variable and machine parameters, reference variables depending on input signals and machine parameters or prediction variables and machine parameters being stored in the database.

7. Method according to claim 4, characterized by generating a control signal when the difference variable reaches, exceeds or falls below a tolerance threshold of the reference variable.

8. Method according to claim 1, characterized by the identification of a portion of the fed input meat product from the input signals by means of a morphology model.

9. Method according to claim 1, characterized in that a portion of the fed input meat product is identified by means of a database depending on the input signals, portion data of meat products depending on input signals being stored in the database.

10. Monitoring apparatus for automatically monitoring an apparatus for processing meat products, in particular fish, characterized by a prediction unit for determining a yield-relevant prediction variable, the prediction unit being connected by means of a data connection with input sensors for acquiring geometric and/or weight data of an input meat product fed to the apparatus for meat processing, a yield determining unit for determining at least one yield-relevant yield variable, the yield determining unit being connected to the output sensors for acquiring geometric and/or weight data of a meat product yield by means of a further data connection, and a difference unit for calculating a difference variable from the difference between one of the prediction variables and the at least one corresponding yield variable, the difference unit being connected to the prediction unit by a further data connection and the difference unit being connected to the yield determining unit by a further data connection.

11. Monitoring apparatus according to claim 10, characterized in that the prediction unit has a morphology model for calculating the prediction variable from the input signals or the prediction unit has a morphology model and a machine model for calculating the prediction variable from the input signals and the machine parameters, a machine parameter memory being connected to the prediction unit by means of a further data connection for storing machine parameters.

12. Monitoring apparatus according to claim 10, characterized in that a database in which prediction variables are stored depending on input signals or input signals and machine parameters, is connected to the prediction unit by means of a further data connection, and the prediction unit is designed to assign a corresponding value from the database to the prediction variable depending on the input signals or the input signals and machine parameters.

13. Monitoring apparatus according to claim 10, characterized by a comparison unit for determining a comparison variable by comparing the difference variable with an optionally predetermined, calculated or assigned reference variable, the comparison unit being connected by a further data connection to the difference unit.

14. Monitoring apparatus according to claim 13, characterized by a reference variable determining unit with a further machine model and/or morphology model for calculating the reference variable from the input signals and the machine parameters or from the prediction variable and the machine parameters by means of the machine model and/or the morphology model, the input sensors, the machine parameter memory and the comparison unit in each case being connected by a further data connection to the reference variable determining unit.

15. Monitoring apparatus according to claim 13, characterized in that a reference variable determining unit is designed to assign a corresponding value from a database to the reference variable depending on the input signals, the prediction variable and/or the machine parameters, reference variables depending on input signals, prediction variables and/or machine parameters being stored in the database, and the input sensors, the prediction unit and/or the machine parameter memory in each case being connected by means of a further data connection to the reference variable determining unit.

16. Monitoring apparatus according to claim 13, characterized by a control signal determining unit for producing a control signal when the difference variable reaches, exceeds or falls below a tolerance threshold of the reference variable, the difference unit or the difference unit and the reference variable determining unit in each case being connected by means of a further data connection to the control signal determining unit.
